(19) United States
(12) Patent Application Publication
(10) Pub. No.: US 2024/0383832 A1
(43) Pub. Date: Nov. 21, 2024

(54) Process for preparing an acetal from an olefin using PtI2 or PtBr2

(71) Applicant: Evonik Oxeno GmbH & Co. KG, Marl (DE)

(72) Inventors: Robert Franke, Marl (DE); Carolin Schneider, Monheim am Rhein (DE); Ralf Jackstell, Rostock (DE); Matthias Beller, Ostseebad Nienhagen (DE)

(73) Assignee: Evonik Oxeno GmbH & Co. KG, Marl (DE)

(21) Appl. No.: 18/649,655

(22) Filed: Apr. 29, 2024

(30) Foreign Application Priority Data
May 16, 2023 (EP) 23173654.7

(51) Int. Cl. C07C 41/54

(52) U.S. Cl. CPC C07C 41/54

(57) ABSTRACT

Process for preparing an acetal from an olefin using PtI₂ or PtBr₂.

The present invention relates to a process for preparing an acetal from an olefin using PtI₂ or PtBr₂.

It was an object of the present invention to provide a process for preparing an acetal from an olefin. The intention here is to achieve a good yield.

This object is achieved by a process according to claim 1.

Process comprising the process steps of:

a) initially charging an olefin;

b) adding a compound of formula (I):

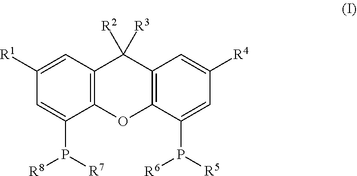

where R¹, R², R³, R⁴, R⁵, R⁶, R⁷, R⁸ are selected from: —H, —(C₁-C₁₂) -alkyl, - (C₆-C₂₀)-aryl; and, if R¹, R², R³, R⁴, R⁵, R⁶, R⁷, R⁸ are —(C₆-C₂₀)-aryl, the aryl ring may have substituents selected from: —(C₁-C₁₂)-alkyl, —O—(C₁-C₁₂)-alkyl;

c) adding PtI₂ or PtBr₂;

d) adding an alcohol selected from: methanol, ethanol, 1-propanol, 1-butanol, 1-pentanol, 1-hexanol, 1-heptanol, 1-octanol, ethane-1,2-diol, propane-1,2-diol, propane-1,3-diol, butane-1,4-diol;

e) feeding in CO and H₂;

f) heating the reaction mixture from steps a) to e), to convert the olefin to an acetal.

In this process, process steps a) to e) can be effected in any desired sequence. Typically, however, CO and H₂ are added after the co-reactants have been initially charged in steps a) to d).

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

Suitable (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression (C₆-C₂₀)-aryl encompasses mono-or polycyclic aromatic hydrocarbyl radicals having 6 to 20 carbon atoms. These are preferably (C₆-C₁₄)-aryl, more preferably (C₆-C₁₀)-aryl.

Suitable (C₆-C₂₀)-aryl groups are especially phenyl, naphthyl, indenyl, fluorenyl, anthracenyl, phenanthrenyl, naphthacenyl, chrysenyl, pyrenyl, coronenyl. Preferred (C₆-C₂₀)-aryl groups are phenyl, naphthyl and anthracenyl.

In one variant of the process, R², R³, R⁵, R⁶, R⁷, R⁸ are selected from: —(C₁-C₁₂)-alkyl, —(C₆-C₂₀)-aryl.

In one variant of the process, R⁵, R⁶, R⁷, R⁸ are —(C₆-C₂₀)-aryl.

In one variant of the process, R⁵, R⁶, R⁷, R⁸ are —Ph.

In one variant of the process, R² and R³ are —(C₁-C₁₂)-alkyl.

In one variant of the process, R² and R³ are —CH₃.

In one variant of the process, R¹ and R⁴ are —H.

In one variant of the process, the compound (I) has the structure (1):

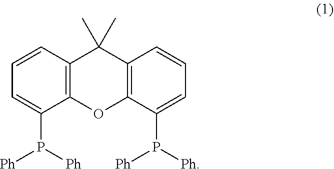 [SMILES: CC1(C)c2cccc(P(c3ccccc3)c3ccccc3)c2Oc2c(P(c3ccccc3)c3ccccc3)cccc21]

In one variant of the process, PtI₂ is added in process step c).

In one variant of the process, PtBr₂ is added in process step c).

In one variant of the process, the alcohol in process step d) is selected from: methanol, ethanol, 1-propanol, 1-butanol, ethane-1,2-diol, propane-1,2-diol.

In one variant of the process, the alcohol in process step d) is MeOH.

In one variant of the process, CO and H₂ are fed in at a pressure in a range from 1 MPa (10 bar) to 6 MPa (60 bar).

In one variant of the process, CO and H₂ are fed in at a pressure in a range from 3 MPa (30 bar) to 5 MPa (50 bar).

In one variant of the process, the olefin is selected from: ethene, propene, 1-butene, cis- and/or trans-2-butene, isobutene, 1,3-butadiene, 1,2-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, tri-n-butene, 1,7-octadiene, 1,9-decadiene, methyl 9-decenoate (9-Dame) or mixtures thereof.

In one variant of the process, the olefin has two double bonds.

In one variant of the process, the olefin has two terminal double bonds.

In one variant of the process, the olefin is 1,3-butadiene.

In one variant of the process, the reaction mixture is heated to a temperature in the range from 50° C. to 150° C.

In one variant of the process, the reaction mixture is heated to a temperature in the range from 80° C. to 140° C.

In one variant of the process, this process comprises the additional process step e′): e′) adding a solvent that is not an alcohol.

In one variant of the process, the solvent is selected from: THF, MTBE, DCM, ACN, heptane, DMF, toluene, xylene, mesitylene, dibenzyltoluene.

The invention is to be elucidated in detail hereinafter with reference to working examples.

### GENERAL EXPERIMENTAL DESCRIPTION

The olefin, an inert solvent, the alcohol, PtX₂ (X=halogen) and the ligand are placed in a stainless steel autoclave from Parr Instruments under argon. Synthesis gas CO/H₂ (1:1) is injected and the reaction is conducted at the chosen reaction temperature while stirring. After the end of the reaction time, the autoclave is cooled down to room temperature, the residual pressure is released, and a GC sample for determination of the yield of target product is taken and analysed.

### Variation of the Olefin: 1-Octene

### Reaction Conditions:

10 mmol 1-octene, 0.5 mol % PtI₂, 2.2 eq. Xantphos (1), solvent: toluene, p(CO/H₂): 40 bar, T: 80° C., t: 24 h.

Monoalcohol: 4 equivalents: diol: 2 equivalents

| Acetal | Yield [%] |
|---|---|
| 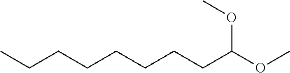 | 90 |
| 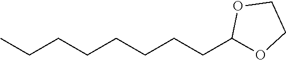 | 48 |
| 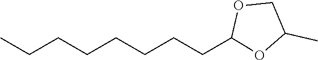 | 75 |
| 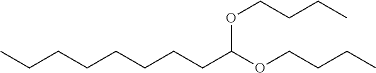 [SMILES: CCCCCCCCC(OCCCC)OCCCC] | 81 |

### Variation of the Alcohol: 1,3-Butadiene

### Reaction Conditions:

10 mmol 1,3-butadiene, 0.5 mol % PtI₂, 2.2 eq. Xantphos (1), solvent: toluene, p (CO/H₂): 40 bar, T: 80° C., t: 24 h.

Monoalcohol: 4 equivalents: diol: 2 equivalents

| | Yield |
|---|---|
| Acetal | [%] |
| 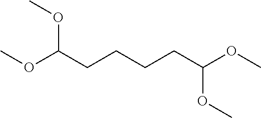 | 55 |
| 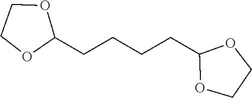 [SMILES: C(CCC1OCCO1)CC1OCCO1] | 86 |
| 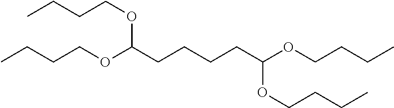 [SMILES: CCCCOC(CCCCC(OCCCC)OCCCC)OCCCC] | 50 |

### Variation of the Halogen

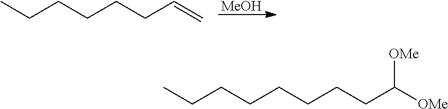

10 mmol of 1-octene, 40 mmol of methanol, 10 ml of toluene, PtX₂ (0.1 mol %), Xantphos (1) (0.22 mol %) are introduced into a 25 ml autoclave under argon. Synthesis gas (CO/H₂=1.1) is injected to 40 bar. The reaction is conducted at 120° C. while stirring and while measuring the gas consumption in the autoclave (electronic pressure transducer, Specview software) for 24 h. The autoclave is cooled down to room temperature and the pressure is released. The yield is determined by taking a GC sample.

The reaction is conducted for X=I/Br/Cl.

### Reaction Conditions:

10 mmol 1-octene, 0.1 mol % PtX₂, 2.2 eq. Xantphos (1), solvent: toluene, p (CO/H₂): 40 bar, T: 120°° C., t: 24 h.

### Yields:

PtI₂: 46%

PtBr₂: 36%

PtCl₂*: 7%

* noninventive comparative experiment

As shown by the experimental results, the object is achieved by the process according to the invention.

1. Process comprising the process steps of:
a) initially charging an olefin;
b) adding a compound of formula (I): 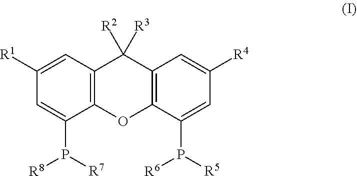
where R¹, R², R³, R⁴, R⁵, R⁶, R⁷, R⁸ are selected from: —H, —(C₁-C₁₂)-alkyl, —(C₆-C₂₀)-aryl;
and, if R¹, R², R³, R⁴, R⁵, R⁶, R⁷, R⁸ are —(C₆-C₂₀)-aryl, the aryl ring may have substituents selected from: —(C₁-C₁₂)-alkyl, —O—(C₁-C₁₂)-alkyl;
c) adding PtI₂ or PtBr₂;
d) adding an alcohol selected from: methanol, ethanol, 1-propanol, 1-butanol, 1-pentanol, 1-hexanol, 1-heptanol, 1-octanol, ethane-1,2-diol, propane-1,2-diol, propane-1,3-diol, butane-1,4-diol;
e) feeding in CO and H₂;
f) heating the reaction mixture from steps a) to e), to convert the olefin to an acetal.

2. Process according to claim 1, where R², R³, R⁵, R⁶, R⁷, R⁸ are selected from: —(C₁-C₁₂)-alkyl, —(C₆-C₂₀)-aryl.

3. Process according to claim 1, where R⁵, R⁶, R⁷, R⁸ are —(C₆-C₂₀)-aryl.

4. Process according to claim 1, where R² and R³ are —(C₁-C₁₂)-alkyl.

5. Process according to claim 1, where R¹ and R⁴ are —H.

6. Process according to claim 1, wherein the compound (I) has the structure (1):

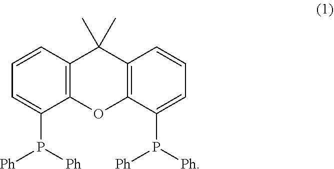 [SMILES: CC1(C)c2cccc(P(c3ccccc3)c3ccccc3)c2Oc2c(P(c3ccccc3)c3ccccc3)cccc21]

7. Process according to claim 1, wherein PtI₂ is added in process step c).

8. Process according to claim 1, wherein PtBr₂ is added in process step c).

9. Process according to claim 1, wherein the alcohol in process step d) is selected from: methanol, ethanol, 1-propanol, 1-butanol, ethane-1,2-diol, propane-1,2-diol.

10. Process according to claim 1, wherein the alcohol in process step e) is MeOH.

11. Process according to claim 1, wherein CO and H₂ are fed in at a pressure in a range from 1 MPa (10 bar) to 6 MPa (60 bar).

12. Process according to claim 1, wherein CO and H₂ are fed in at a pressure in a range from 3 MPa (30 bar) to 5 MPa (50 bar).

13. Process according to claim 1, wherein the olefin is selected from: ethene, propene, 1-butene, cis- and/or trans-2-butene, isobutene, 1,3-butadiene, 1,2-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, tri-n-butene, 1,7-octadiene, 1,9-decadiene, methyl 9-decenoate (9-Dame) or mixtures thereof.

14. Process according to claim 1, wherein the olefin has two double bonds.

15. Process according to claim 1, wherein the olefin has two double terminal bonds.
